(19) United States
(12) Patent Application Publication
(10) Pub. No.: US 2005/0015126 A1
(43) Pub. Date: Jan. 20, 2005

(54) Personal thermotherapy instrument

(76) Inventor: Hong-Jik Doo, Seoul (KR)

Correspondence Address: Stanzione & Kim, LLP, 1st. fl., 1740 N Street, NW, Washington, DC, 20036, US

(21) Appl. No.: 10/773,242

(22) Filed: Feb. 9, 2004

(30) Foreign Application Priority Data
Feb. 25, 2003 (KR) 10-2003-11732
Mar. 6, 2002 (KR) 10-2003-14033
May 22, 2003 (KR) 10-2003-32586

(51) Int. Cl. A61F 7/00

(52) U.S. Cl. 607/108; 607/112

(57) ABSTRACT

A personal thermotherapy instrument formed by fastening and fixing as one body is provided with a collapsible folding type frame to a lower part of a thermotherapy system so that the thermotherapy system can be stored in a folded position by the folding type frame. The thermotherapy is provided with a separation unit to externally expose only thermotherapy components, such as a spinal corrector and a foot pressure plate provided on the folding type frame, in performing an after-sale service.

### CROSS-REFERENCE TO RELATED APPLICATIONS

This application claims the benefit of: Korean Patent Application No. 10-2003-11732, filed with the Korea Industrial Property Office on Feb. 25, 2003; Korean Patent Application No. 10-2003-14033, filed with the Korea Industrial Property Office on Mar. 6, 2003; and Korean Patent Application Mo. 10-2003-32586, filed with the Korea Industrial Property Office on May 22, 2003, the disclosures of which are incorporated herein by reference.

### BACKGROUND OF THE INVENTION

1. Field of the Invention

The present invention relates to a personal treatment instrument, and more particularly, to a personal thermotherapy instrument, which can be stored in a folded position, by applying a folding type frame to a thermotherapy system used as a bed type instrument, and can be easily used in performing an assembling work and an after-sale service by varying an assembling structure of the thermotherapy system.

2. Description of the Related Art

A recent rapid increase in the living standard brings about an increased concern for health. This increased concern for health has resulted in an invention for personal thermotherapy instruments to provide pressurization and fermentation and a spinal correction. Research to more efficiently use the thermotherapy instrument is being progressed at the present time.

Describing in brief an operational principle and configuration with an example, a general thermotherapy instrument A (Korea Registration Utility Model Publication No. 0292042) is divided into an upper therapy mat 10 and a lower therapy mat 20 as shown in FIG. 10. On the upper therapy mat 10, the upper body of a user is positioned, and on the lower therapy mat 20, the lower body of the user is positioned. The upper therapy mat and the lower therapy mat are connected integrally with each other, and are put on a frame 30 so as to be used as a bed type instrument.

On the upper therapy mat 10, a spinal corrector 11 is installed in a length direction, to move forward and backward a therapy director 14 including a roller 14a or pressure bead 14b shaped member, and on the lower therapy mat 20, a foot pressure plate 21 is formed including a thermo-pressure function for the sole of the foot. The spinal corrector 11 is configured in such a way that when a reversible motor 12 fixedly installed in one side interior of the upper therapy mat 10 is driven, a drive transfer device 13 such as a belt, a chain, a wire, etc., connected to that motor 12, moves up and down along the length of the upper therapy mat 10 to thus move up and down the therapy director 14.

When the user manipulates the system under such a state that the user lies on a thermotherapy system A, the spinal director 14 in contact with a user's spine moves every joint of the spine of the user to pressure and ferment the spine of the user. Herewith, the therapy director 14 is used selectively as the roller 14a or pressure bead 14b shaped member through a selection of the user.

The foot pressure plate 21 is utilized by vertically standing up the laid foot pressure plate and then by closely sticking the sole of the foot of the user onto a surface of the foot pressure plate 21.

This thermotherapy system A of the prior art has an advantage of pressurizing and fermenting the spine of the user, thereby correcting the spine. However, since most thermotherapy systems A are configured while giving an importance on a function of efficiently treating the spine, to be used in the form of a bed type instrument fixed to the frame 30 of a single body, there are problems as follows.

First, the manufactured frame 30 has the bed type shape, thus occupying much movement space. That is, in transferring the product, the frame must be disassembled piece by piece, and then the disassembled components must be re-assembled and again installed at its original location. This not only causes an inconvenience for users, but also requires a considerable amount of manpower.

Next, the thermotherapy system A assembled as the bed type instrument does not have a specific foldable providing function, and therefore a considerable amount of installation space is needed such that the space in which the bed type instrument is kept is not efficiently utilized.

Furthermore, an internal structure of the upper therapy mat 10 and the lower therapy mat 20 in the prior art of FIG. 10 is configured by respectively arranging plywood of a plate shape and urethane, installing and fixing the spinal corrector 11 and the foot pressure plate 21, serving as the thermotherapy components etc., to the arranged plywood, and by placing as one piece the arranged urethane onto an edge of the plywood and simultaneously covering the entire arrangement of the urethane and plywood with leather. That is, the thermotherapy instrument of the prior art must be manufactured by individually and specifically manufacturing thermotherapy components, such as urethane and leather, and then assemble them one by one, which causes not only an increase in manufacturing costs, but also a decrease in productivity due to complications in the assembling work.

In particular, in the case of an after-sale service of the instrument being required, the leather must be peeled off from the thermotherapy instrument, and the urethane and electric field parts must be separated piece by piece, and then the thermotherapy components are exposed externally. After this, the after-sale service can be executed. Thus, there are many inconvenient problems in reinstalling the instrument after completing the after-sale service of the instrument.

### SUMMARY OF THE INVENTION

A feature of the present invention is to provide a personal thermotherapy instrument capable of ensuring an installation space and increasing a spatial utility when not used, by applying a folding type frame to a thermotherapy instrument having a bed type shape.

Another feature of the present invention is to provide a personal thermotherapy instrument capable of curtailing a cost of production and simultaneously providing a simple executable assembly and simple after-sale service by improving an assembling structure of the thermotherapy instrument to provide pressure and fermentation to the spine of the user.

Additional aspects and/or advantages of the invention will be set forth in part in the description which follows and, in part, will be obvious from the description, or may be learned by practice of the invention.

The foregoing and/or other features of the present invention are achieved by providing a personal thermotherapy instrument that can be stored in a folded position, by fastening and fixing a collapsible folding type frame to a lower part of the thermotherapy instrument, in which only thermotherapy components, such as a spinal corrector or a foot pressure plate, etc., provided on the folding type frame, are exposed in performing an after-sale service, by equipping a separation unit with this thermotherapy instrument.

### BRIEF DESCRIPTION OF THE DRAWINGS

These and/or other aspects and advantages of the present invention will become apparent and more readily appreciated from the following description of the embodiments, taken in conjunction with the accompanying drawings of which:

FIG. 1 is a perspective view of a thermotherapy instrument according to an embodiment of the present invention;

FIG. 2 is a sectional view of the thermotherapy instrument of FIG. 1;

FIG. 3 is a perspective view illustrating a separated state of the thermotherapy instrument of FIG. 1 provided on a folding type frame according to the present invention;

FIG. 4 is a perspective view showing a separated state of the thermotherapy instrument of FIG. 1;

FIG. 5 is a perspective view of the folding type frame according to FIGS. 1 and 3;

FIG. 6 is a perspective view illustrating main components of a folding preventing part and a hand danger preventing plate of the thermotherapy instrument of FIG. 1;

FIG. 7 is a perspective view showing main parts of a fold leg according to the thermotherapy instrument of FIG. 1;

FIG. 8 is a front view of the thermotherapy instrument illustrated in FIG. 1;

FIG. 9 is a front view of the thermotherapy instrument illustrating a folded state of the thermotherapy instrument through use of the folding type frame illustrated in FIGS. 1 and 3; and

FIG. 10 is a perspective view of a conventional thermotherapy instrument.

### DETAILED DESCRIPTION OF THE PREFERRED EMBODIMENTS

The present invention and exemplary embodiments thereof are more fully described below with reference to the accompanying drawings. This invention may, however, be embodied in many different forms and should not be construed as being limited to the exemplary embodiments set forth herein; rather, these exemplary embodiments are provided so that this disclosure is thorough and complete, and conveys the concept of the invention to those skilled in the art. In the inventive description, details of widely known functions of constructions will be omitted for the sake of brevity.

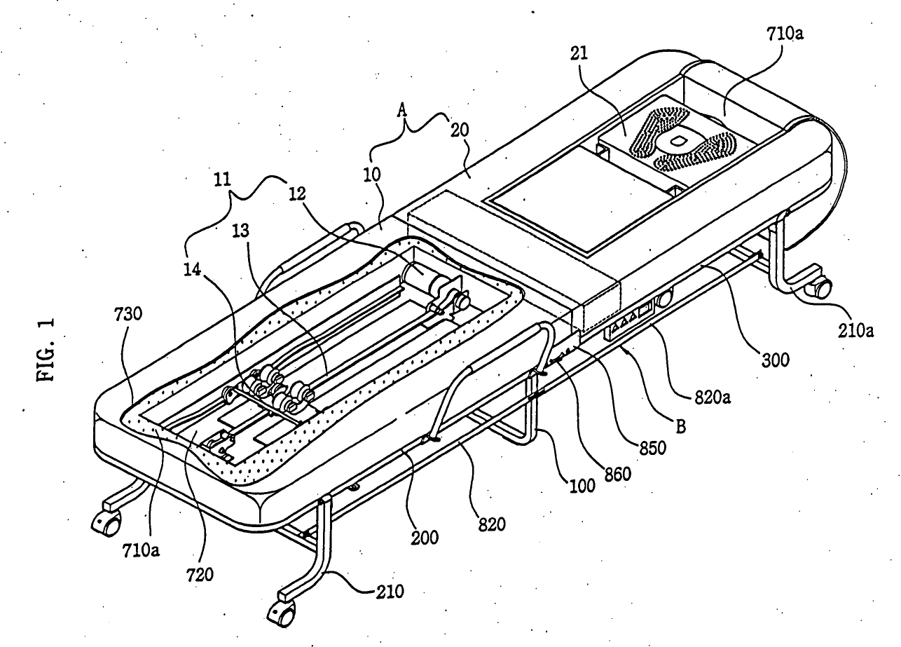

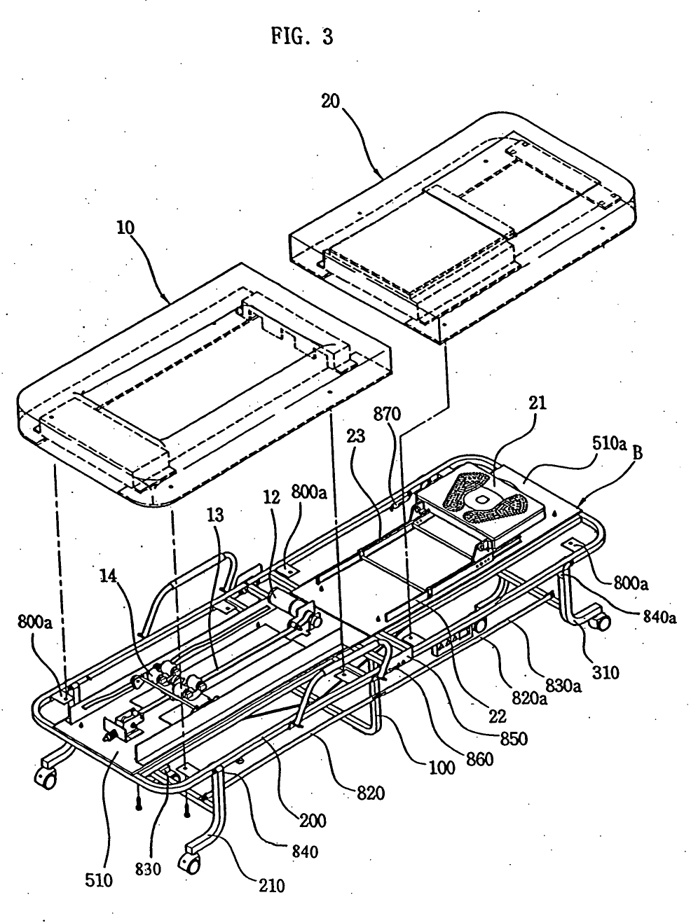

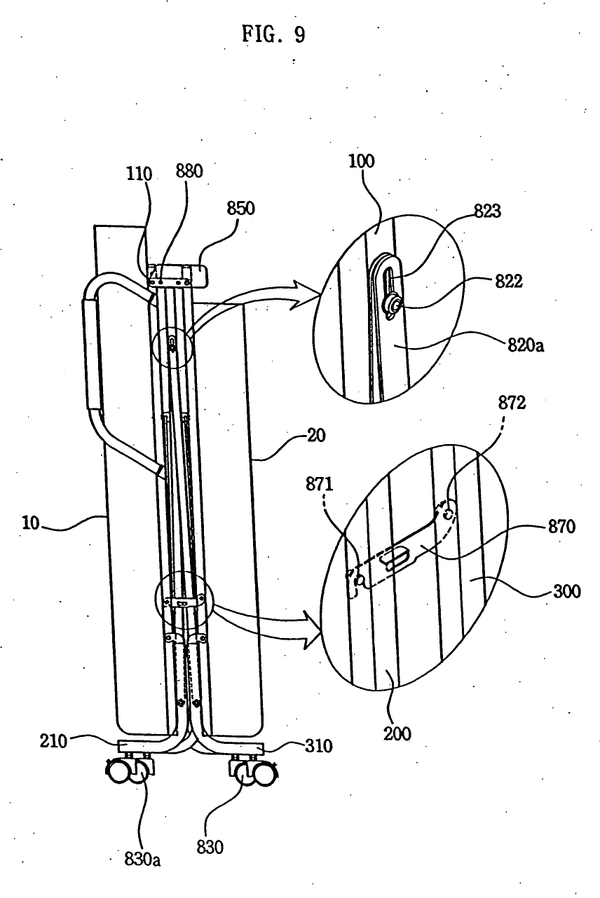

FIG. 1 is a perspective view of a thermotherapy instrument according to the present invention. References to the present invention will be made with respect to FIGS. 1 through 9.

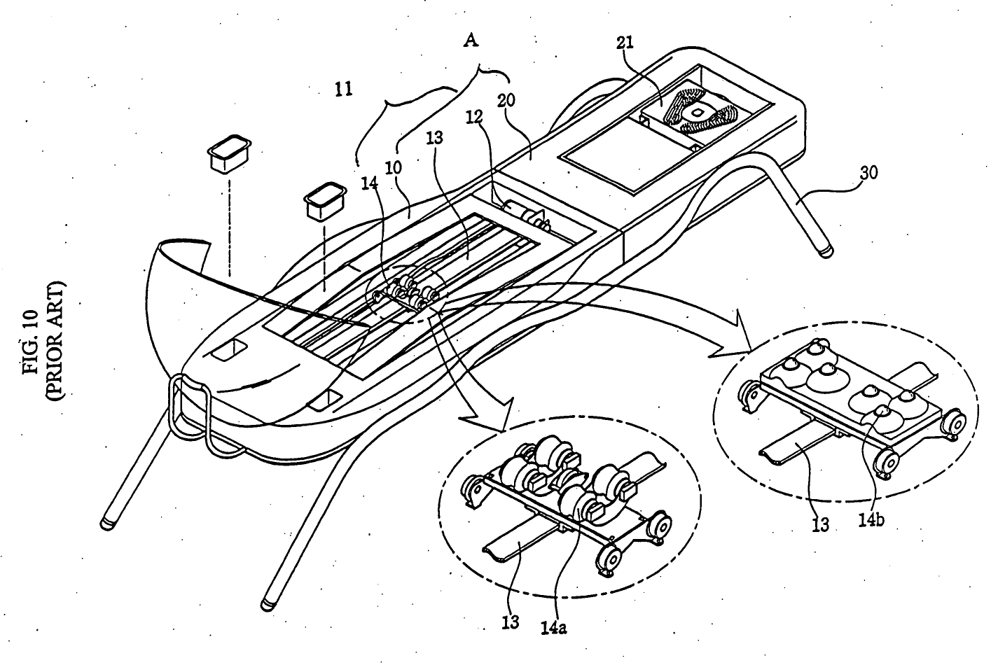

The present invention has a similar technique as the prior art shown in FIG. 10 from the viewpoint of a basic line used as a bed type instrument, in which provided are an upper therapy mat 10 on which a spinal corrector 11 is installed to move forward and backward a therapy director 14 having a roller or pressure bead shape member and on which the upper body of a user is positioned; and an lower therapy mat 20 on which the lower body of the user is positioned and afoot pressure plate 21 is installed. A detailed description will be omitted since the invention has a generally used circuit relationship and peripheral technique by operating the therapy director 14 through the foot pressure plate 21 and the spinal corrector 11.

The present invention has the following detailed improved configuration with respect to the comfiguration of the conventional device shown in FIG. 10.

According to an exemplary embodiment of the invention, a collapsible function is added to a personal thermotherapy system A used as a bed type instrument, so as to ensure sufficient installation space and to simply perform an assembling and after-sale service work. As shown in FIGS. 1 through 9, a collapsible folding type frame B is fastened and fixed in one body to a lower part of a thermotherapy system A, to be stored in a folded position through use of the folding type frame B. Further, a separation unit C is equipped with the thermotherapy instrument so that thermotherapy therapy components 520 and 520a provided on the folding type frame B are exposed externally when executing the after-sale service work.

In order to connect as one body and to fix the thermotherapy system A to an upper part of the folding type frame B, the thermotherapy instrument requires a folding unit through which the thermotherapy instrument can be kept in the folded state by using the folding type frame B.

The folding type frame B used as a folding unit is largely classified into a middle frame 100 that is installed vertically in the middle thereof and serves as a middle leg role to support a folding portion provided in an unfolded state of the instrument; and an upper frame 200 and a lower frame 300, which are separated into two sides by a center on the middle frame 100, and which are individually hinge (110)-combined at the two sides of an upper part of the middle frame 100. The upper and lower frames 200 and 300 are configured to be each foldable downward centering on the middle frame 100 and to be stood vertically.

Both ends of springs 400 and 410 are connected between a lower part of the middle frame 100 and an upper part of the upper and lower frames 200 and 300, respectively, to thus smoothen a folding operation through the springs 400 and 410 when the thermotherapy system A is folded or unfolded.

Herewith, a spring force of the springs 400 and 410 allows the thermotherapy system A to be easily foldable even though little force is applied to the upper frame 200 and the lower frame 300 when the thermotherapy system A is being folded. If the thermotherapy instrument is being placed in an unfolded state, the springs 400 and 410 provide a proper spring force to prevent the upper and lower frames 200 and 300 from being unfolded suddenly.

In the configuration of the middle frame 100, a middle sub frame 120 having a predetermined length is equipped with an upper portion of the middle frame 100, and in the left and right ends of the middle sub frame 120 a folding part 420 is adapted to hinge (110) combine the upper frame 200 and the lower frame 300. In a lower part of the middle sub frame 120, a support frame 130 is equipped as a single body to support a folding portion in a contact state with the thermotherapy system A when it is unfolded.

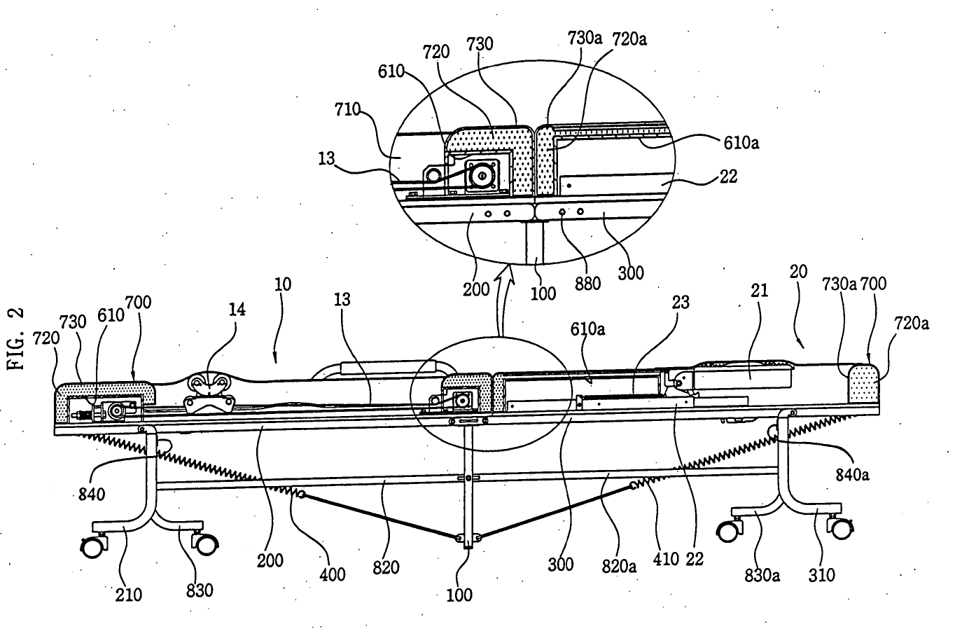

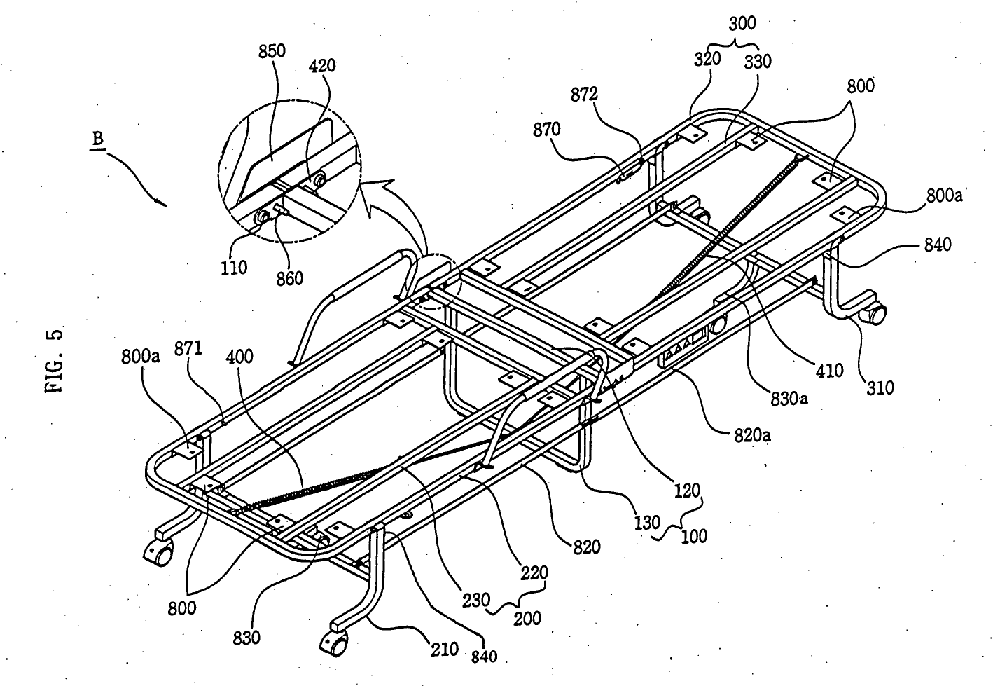

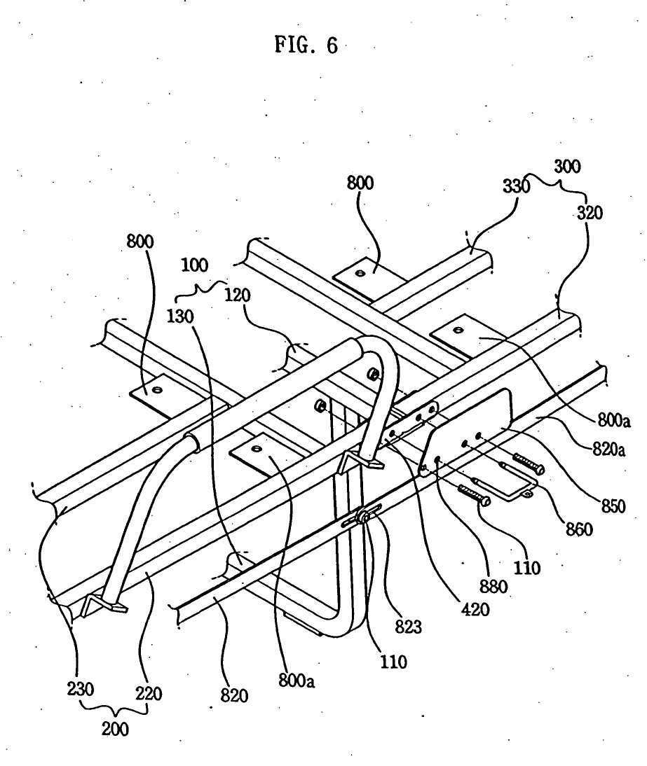

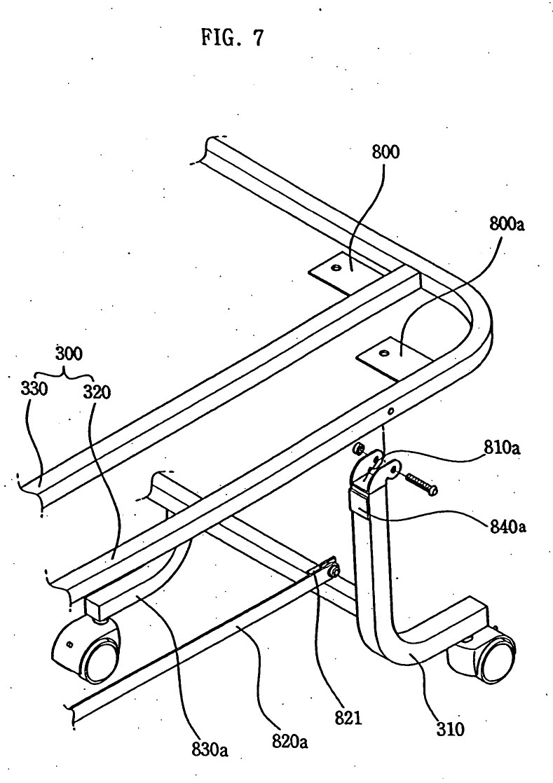

To more conveniently and stably utilize the thermotherapy system A, the folding type frame B has an additional installation feature of fastening plates 800 and 800a (see, for example, FIGS. 5 and 6), stable mounting parts 810 and 810a (see FIG. 7), connection links 820 and 820a, auxiliary legs 830 and 830a (see, for example FIG. 2), noise absorption pads 840 (see, for example, FIG. 5) and 840a (not shown), a hand danger preventing plate 850, a folding preventing part 860 (FIG. 5), and a stop hook 870 (FIG. 5).

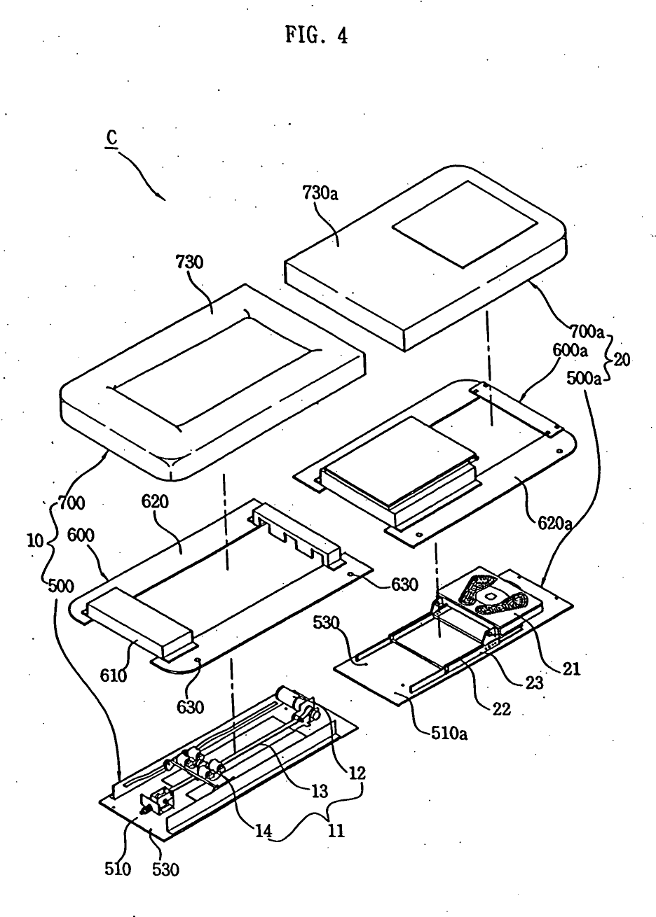

The fastening plates 800 and 800a (see, for example, FIG. 6) are each equipped on outer frames 220 and 320 and inner frames 230 and 330 provided with an upper part of the folding type frame B, and on the fastening plates 800 and 800a, side plates 620 and 620a (see, for example, FIG. 4) and base plates 510 and 510a (FIG. 4) of the thermotherapy system A are each fastened and fixed thereto.

The stable mounting parts 810 (not shown) and 810a are formed on an upper side of the fold legs 210 and 310, respectively, and are hinge-combined with the upper and lower sides of the folding type frame B. When the folding type frame B is unfolded, lower parts of the outer frames 220 and 320 closely adhere onto upper parts of the stable mounting parts 810 and 810a, respectively, to thus entirely support the folding type frame B. That is, this arrangement is provided to prevent the load from being concentrated onto the hinge (110) side of the fold legs 210 and 310 when using the thermotherapy instrument.

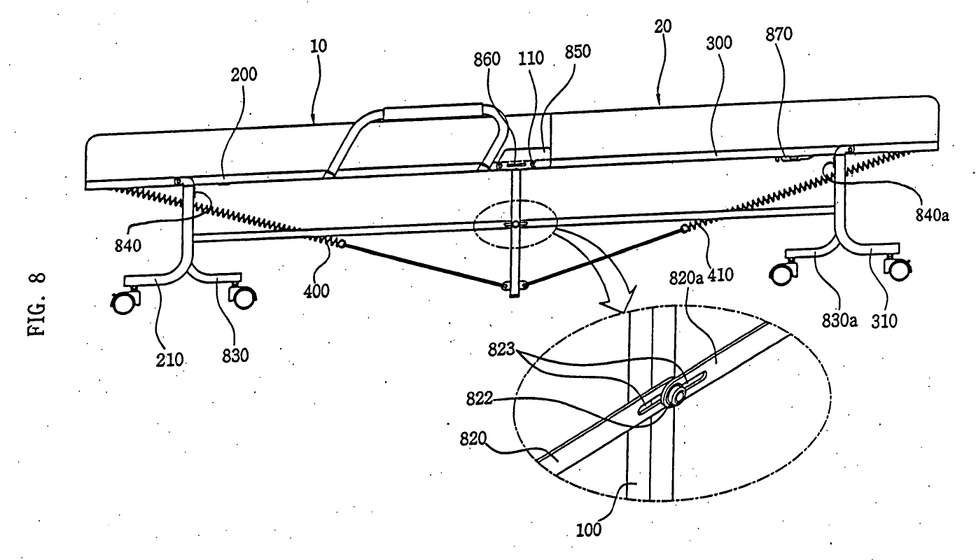

The connection links 820 and 820a each have a predetermined length and are connected between the fold legs 210 and 310 and the middle frame 100 to prevent the fold legs 210 and 310 from being bent outward when using the thermotherapy instrument. In other words, one end of the connection link is hinge connected with a bracket 821 (see, for example, FIG. 7) formed on the fold legs 210 and 310, and another end of the connection link is connected with a connection pin 822 (see, for example, FIG. 8) projecting from the middle frame 100.

In particular, long flutes 823 (FIG. 8) are each formed in portions and connected with the connection pin 822 so that the connection links 820 and 820a can be varied along each of the long flutes 823, to thus smoothly perform a folding function when the thermotherapy instrument is folded or unfolded.

The auxiliary legs 830 and 830a are each positioned in the middle of a lower part of each fold leg 210 and 310, respectively, and are formed in an inward direction opposite to a direction of the fold legs 210 and 310, respectively. The auxiliary legs are formed at mutually crossed positions so as not to be bumped against each other when folding the thermotherapy instrument.

The noise absorption pad 840 (FIG. 7) is equipped with a sidewall of the fold leg, and absorbs noise generated when the fold leg 210 and the fold leg 310 are bumped under the folding operation.

The hand danger preventing plate 850 is formed into a plate shape, and is integrated into an outer side of the folding part 420 (FIG. 5) of the folding type frame B, and has a predetermined height to prevent the hand of a user from being inserted into a space formed from the folding part 420 when the folded thermotherapy system A is unfolded.

The folding preventing part 860 is formed in a ‘ [symbol] ’ shape and is inserted into an insertion flute 880 that is formed piercing through the hand danger preventing plate 850, the folding part 420 and the outer frames 220 and 230 to prevent the unfolded folding type frame B from being suddenly folded by a tension force of the springs 400 and 410.

In contrast, as a unit to prevent the folded folding type frame B from being unfolded to the upper and lower sides by an external interference, any one of the outer frames 220 and 320 has a stop pin 871 (FIG. 5) formed projecting outwardly, and the other of the outer frames has a stop hook 870 that rotates centering on a hinge 872 and is stopped by the stop pin 871, thus preventing the folded folding-type frame B from being unfolded.

In the meantime, describing the separation unit C used as a unit to provide simply assembly and disassembly of the thermotherapy system A from the folding type frame B, first the upper therapy mat 10 and the lower therapy mat 20 are each classified into first assemblies 500 and 500a, middle assemblies 600 and 600a, and second assemblies 700 and 700a (see, for example, FIG. 4). The first assemblies 500 and 500a are always fixed to the folding type frame B, and the middle assemblies 600 and 600a are each, as one unit, connected with the second assemblies 700 and 700a, thus providing a basic configuration separated as one unit from the folding type frame.

The first assembly 500 of the upper therapy mat 10 is provided as one configuration body by installing the spinal corrector 11 comprising well-known thermotherapy components, such as the therapy director 14, a drive transfer apparatus 13 and a reversible motor 12, on an upper part of the base plate 510 formed in a plate shape.

The medium assembly 600 is provided with a front housing 610 that is formed with front and back faces thereof to cover the spinal corrector 11, and is combined as one body with the second assembly 700.

The second assembly 700 (see, for example, FIG. 2) is provided as another configuration body and is covered with a leather 730 and a surface of urethane 720 having a space (710) formed in a middle thereof so as to operate the known spinal corrector 11, and is configured to be separated along with the medium assembly 600 in the after-sale service so as to expose the spinal corrector 11 externally.

The first assembly 500a of the lower therapy mat 20 is provided as one configuration body by installing the foot pressure plate 21 as the well-known thermotherapy component comprising a guide rail 22 and a return spring 23 and so on, on an upper part of the base plate 510a formed in a plate shape.

The middle assembly 600a is provided with a front housing 610a (FIG. 2) that covers the guide rail 22 and the return spring 23, etc., the front housing 610a being formed in the front of the middle assembly 600a. The middle assembly 600a is combined with the second assembly 700a as one body.

The second assembly 700a is provided as another configuration body by covering it with leather 730a, a surface of urethane 720a having a space (710a) formation in the middle thereof so as to operate the known foot pressure plate 21, and is configured to be separated along with the middle assembly 600a in performing the after-sale service so as to expose, externally, the foot pressure plate 21 of the first assembly 500a.

Herewith, fastening holes 530 and 630 (FIG. 4) fastened and fixed onto the fastening plates 800 and 800a are each formed on the base plates 510 and 510a, respectively, of the first assemblies 500 and 500a and on the side plates 620 and 620a, respectively, of the medium assemblies 600 and 600a.

In order to apply the present invention based on this configuration, first, individually manufactured are the first assemblies 500 and 500a, in which the well-known thermotherapy components, such as the spinal corrector 11 and the foot pressure plate 21, are each installed on the base plates 510 and 510a, respectively; the middle assemblies 600 and 600a each having the side plates 620 and 620a, respectively, and the front housing 610 and 610a, respectively; and the second assemblies 700 and 700a on which the leather 730 and 730a covers the surface of the urethane 720 and 720a, respectively. Then, the middle assemblies 600 and 600a are assembled as one body with the second assemblies 700 and 700a, respectively.

Subsequently, the first assemblies 500 and 500a each fasten and fix the fastening hole 530 formed in the base plates 510 and 510a to the fastening plate 800 of the inner frames 230 and 330, respectively.

In this state, the second assemblies 700 and 700a are put on an upper part of the first assemblies 500 and 500a, respectively, and then the spinal corrector 11 and the foot pressure plate 21 are individually positioned in the spaces 710 and 710a.

At this time, the fastening holes 630 formed in the side plates 620 and 620a are each screw combined with a respective fastening plate 800a of the outer frames 220 and 320, then the second assemblies 700 and 700a are each combined with the medium assemblies 600 and 600a respectively as one body to then be fastened and fixed to the folding type frame B.

If an after-sale service case occurs under the above described combined state, a worker unfastens a fastening bolt screw-combined with the fastening hole 800a of the side plates 620 and 620a and separates the second assemblies 700 and 700a from the outer frames 220 and 320.

When the worker lifts up the second assemblies 700 and 700a, the middle assemblies 600 and 600a are detached along with the second assemblies 700 and 700a, respectively, from the first assemblies 500 and 500a, and in the detaching procedure, thermotherapy components to be after-sale serviced, namely, the spinal corrector 11 and the foot pressure plate 21, are naturally exposed externally on the folding type frame B. That is, the after-sale service can be performed easily.

Meanwhile, in a state that the thermotherapy system A is combined as one body with the folding type frame B, when the ‘ [symbol] ’-shaped folding preventing part 860 (FIG. 6) is inserted into the insertion flute 880 formed to be piercing through the hand danger preventing plate 850, the folding part 420 and the outer frames 220 and 320a; even though an interference from an external source is applied thereto or a user leaves the thermotherapy instrument unattended, the folding type frame B is not vertically folded by the tension of the springs 400 and 400a.

The thermotherapy system A connected as one body with the folding type frame B can be used, like a well-known thermotherapy instrument, by manipulating the instrument in its unfolded state. At this time, the fold legs 210 and 310 are not bent outward due to support of the connection links 820 and 820a and the auxiliary legs 830 and 830a even though a user lies on the thermotherapy instrument.

In storing the thermotherapy instrument, the folding preventing part 860 is separated from the insertion flute 880, and then the middle frame 100 is slightly lifted upward to be lifted upward and to rise vertically since each end of the upper frame 200 and the lower frame 300 is hinge (110)-combined with the folding part 420 of the middle frame 100.

At this time, since the springs 400 and 400a always pulled inward by the upper frame 200 and the lower frame 300, the upper frame 200 and the lower frame 300 can be folded naturally even by a small force. Further, even though the fold legs 210 and 310 are bumped with each other under the folded procedure, the noise absorption pad 840 absorbs a bump noise so as not to generate noise. Additionally, the folding type frame can be smoothly folded since the connection links 820 and 820a are moved upward through the long flute 823.

In this folded state, the auxiliary legs 830 and 830a are formed in crossed positions so as not to be bumped from each other, and the stop hook 870 is stopped by the stop pin 871. Thus, the thermotherapy instrument is not broadened to the upper and lower sides even though an external interference is applied thereto.

Even if a hand of user is positioned on the folding part 420 to use the folded thermotherapy instrument, there is no danger for the hand of the user to be inserted into the folding part 420 since the hand danger preventing plate 850, having a determined height, covers the folding part.

As described above, a folding type frame is installed in a thermotherapy instrument used as a bed type instrument, and the thermotherapy instrument is foldable by the folding type frame. Therefore, an installation space can be ensured and therefore efficient utilization of space may be achieved when the instrument is in use.

Further, the folding frame is completely combined with the thermotherapy instrument and is folded, then the instrument is sent to consumers. Further, the thermotherapy instrument can be installed conveniently.

In addition, the thermotherapy instrument to pressurize and ferment a user in order to correct spine of the user is provided through a division and an assembly between a first assembly and a second assembly, therefore a cost of production is reduced and an assembling process becomes convenient.

Additionally, when the second assembly is separated from the first assembly in executing after-sale service work, thermotherapy components installed on the base plate are naturally exposed externally, and the after-sale service work can be performed conveniently without additional separation of parts.

While the present invention has been particularly shown and described with reference to the exemplary embodiments described above, it will be understood by those skilled in the art that these exemplary embodiments do not limit the scope of the present invention. Rather, various changes in form and details may be made without departing from the spirit and scope of the invention as defined by the appended claims and their equivalents.

1. A personal thermotherapy instrument comprising:
a collapsible folding type frame fastened and fixed as one body to a lower part of the thermotherapy instrument,
wherein the thermotherapy instrument includes an upper therapy mat on which a spinal corrector moving forward and backward a therapy director having a roller or pressure bead type shape member is installed, and a lower therapy mat on which a foot pressure plate is installed.

2. The instrument as claimed in claim 2, wherein the thermotherapy instrument is provided with a separation unit to externally expose the spinal corrector and the foot pressure plate provided on the folding type frame.

3. The instrument as claimed in claim 2, wherein the separation unit equipped with the upper therapy mat and the lower therapy mat comprises:
first assemblies each having base plates, and each provided as one configuration body by installing the spinal corrector and the foot pressure plate on an upper part of each of the respective base plates;
middle assemblies each provided with electric field parts to cover the spinal corrector and the foot pressure plate, the electric field parts being individually formed on front and back faces thereof and being each provided with side plates each formed in the left and right sides of the electric field parts; and
second assemblies combined as one body with an upper part of the middle assemblies, and provided as another configuration body, by covering with a leather, a surface of urethane having a space forming an interior portion thereof so that the spinal corrector and the foot pressure plate can operate in the space, the second assemblies being separated from the middle assemblies to expose the spinal corrector and the foot pressure plate of the first assemblies externally.

4. The instrument as claimed in claim 1, wherein the folding type frame comprises:
a middle frame vertically positioned in a middle of the folding type frame, the middle frame supporting a folding part in an unfolded state of the instrument and serving as a medium leg role; and
an upper frame and a lower frame, which are divided into first and second parts, centering on the middle frame, hinge-combined with the folding part and folded downward centering on the middle frame to be stood vertically when folding the instrument.

5. The instrument as claimed in claim 4, wherein the upper frame and the lower frame are each provided with a noise absorption pad equipped on a sidewall of each of fold legs, each formed on lower parts of the upper frame and the lower frame.

6. The instrument as claimed in claim 4, wherein one of the upper frame and the lower frame is provided with a stop pin formed projecting therefrom, and the other one of the upper frame and the lower frame is provided with a stop hook that is rotated centering on a hinge and is hooked onto the stop pin to prevent the thermotherapy instrument from being unfolded suddenly.

7. The instrument as claimed in claim 4, wherein the folding part is provided with a hand danger preventing plate formed on an outer side thereof and has a determined height.

8. The instrument as claimed in claim 4, wherein the middle frame and the fold legs are provided with a connection link therebetween, to prevent the fold legs from being bent outward when using the thermotherapy system.

9. The instrument as claimed in claim 7, wherein the hand danger preventing plate and the folding part are provided with an insertion flute formed in a side thereof, and the insertion flute has an insertion of a folding preventing part.

10. A thermotherapy instrument, comprising:
a folding part and first and second frames rotatably connected to the folding part and moving between a folding state and an unfolded state with respect to the folding part;
a first therapy component disposed on the first frame; and
a second therapy component disposed on the second frame.

11. The instrument as claimed in claim 10, wherein the first and second therapy component are a spinal corrector and a foot pressure plate, respectively.

12. The instrument as claimed in claim 10, wherein the first and second frames comprise first and second legs rotatably mounted on the first and second frames, respectively, and moving between first and second positions when the first and second frames move between the folding state and the unfolded state.
